(19) United States
(12) Patent Application Publication
(10) Pub. No.: US 2020/0359913 A1
(43) Pub. Date: Nov. 19, 2020

(54) SYSTEM, APPARATUS, AND METHODS FOR REMOTE HEALTH MONITORING

(71) Applicants: Alireza GHODRATI, Hopkinton, MA (US); Alireza Mahmoudieh, Granite Bay, CA (US)

(72) Inventors: Alireza GHODRATI, Hopkinton, MA (US); Alireza Mahmoudieh, Granite Bay, CA (US)

(21) Appl. No.: 16/874,303

(22) Filed: May 14, 2020

Related U.S. Application Data
(60) Provisional application No. 62/847,573, filed on May 14, 2019.

(51) Int. Cl. A61B 5/0205; A61B 5/00; A61B 5/16

(52) U.S. Cl. CPC A61B 5/02055; A61B 5/742; A61B 5/746; A61B 5/4806; A61B 2503/08; A61B 5/165; A61B 5/0024; A61B 5/681; A61B 5/4833

(57) ABSTRACT

The present disclosure relates to remote health monitoring of subjects, such as the elderly, senior citizens, individuals having chronic health issues, and/or individuals requiring assistance in managing their health or physiological condition.

### PRIOR APPLICATIONS

This Application claims the benefit of and priority to U.S. Provisional Application No. 62/847,573, filed on May 14, 2019, the teachings of which is incorporated by reference herein in its entirety.

### FIELD

The present disclosure generally relates to remote health monitoring, and more particularly to methods, apparatus, devices, and systems for remotely monitoring health of a subject.

### BACKGROUND

Many elderly and senior citizen individuals often wish to live independently at their own homes. Children, loved ones, and relatives of such individuals may also wish to respect these individuals' wishes for living independently, although worrying about their health and wishing that they could be kept informed of their health status and be alerted if/when help is needed. While existing assistive remote care technologies can provide peace of mind by monitoring these individuals, these technologies are often intrusive (e.g., require use of video cameras) or require a person's active participation in the monitoring (e.g., pressing a button when a fall occurs).

### SUMMARY

The present disclosure relates to remote health monitoring of subjects, such as the elderly, senior citizens, individuals having chronic health issues, and/or individuals requiring assistance in managing their health or physiological condition.

In one aspect, a system for remote health monitoring is featured. The featured system comprises one or more sensors each configured to detect a first plurality of parameters relating to one or more physiological conditions of a subject, an interface configured to receive a second plurality of parameters relating to the physiological condition(s) of the subject, where the second plurality of parameters can be generated by other sensors, and at least one processor coupled to the one or more sensors and the interface. The processor can be configured to monitor the first and second pluralities of parameters and, in an event at least one parameter falls outside of a predetermined range, issue an alarm signal indicating onset of an undesired event in the physiological condition(s) of the subject.

In another aspect, a system for health monitoring comprises one or more interfaces each configured to detect a plurality of parameters relating to physiological condition(s) of a subject and at least one processor coupled to the one or more interfaces. The processor can be configured to monitor the plurality of parameters and, in an event at least one parameter falls outside of a predetermined range, issue an alarm signal indicating onset of an undesired event in the physiological condition(s) of the subject.

In other examples, the aspects above, or any system, method, apparatus described herein can include one or more of the following features.

The at least one interface can comprise a sensor configured to detect at least one parameter relating to the physiological condition(s) of a subject. By way of example, the sensor(s) can comprise at least one of a heart rate detection sensor, a location determination sensor, a movement sensor, and a sensor connected to one or more medicine containers storing medication consumed by the subject. Further, the parameters can include information relating to at least one of medication intake by the subject, sleeping patterns of the subject, physical activity rate of the subject, blood pressure of the subject, weight of the subject, blood glucose level of the subject, physical state of the subject, and emotional state of the subject.

The predetermined range can comprise an expected range of values for the first and the second plurality of parameters. The processor can be configured to determine the expected range based on historical measures of the at least one parameter. For example, the processor can be configured to obtain the historical measures of the at least one parameter by storing measurement of the at least one parameter at two or more instants of time. Additionally or alternatively, the processor can be configured to determine a subject-specific expected range for the subject based on the historical measures. Further, the processor can be configured to monitor the parameters and, in an event the pluralities of parameters fall within the predetermined range, issue an all clear signal indicating that the subject is in a stable physiological condition. In some embodiments, the processor can be configured to monitor the at least one parameter over a predetermined time interval.

In some embodiments, the processor can be configured to issue the alarm signal to an entity capable of providing immediate assistance to the subject. Alternatively or additionally, the alarm signal can be a reminder signal issued to the subject.

Further, in some embodiments, the processor can be configured to monitor the at least one parameter over a predetermined time interval. The predetermined time interval can comprise at least one time interval during which the one or more sensors did not operate to detect the first plurality of parameters, and the processor can be configured to estimate values of the plurality of parameters during the at least one time interval. The processor can also be configured to determine a confidence interval for estimated values of the plurality of parameters and in an event at least one estimated value of a parameter falls outside of the predetermined range, issue the alarm signal.

Other aspects and advantages of the invention can become apparent from the following drawings and description, all of which illustrate the various aspects of the invention, by way of example only.

### BRIEF DESCRIPTION OF THE DRAWINGS

A detailed description of various embodiments is provided herein below with reference, by way of example, to the following drawings. It will be understood that the drawings are exemplary only and that all reference to the drawings is made for the purpose of illustration only, and is not intended to limit the scope of the embodiments described herein below in any way. For convenience, reference numerals may also be repeated (with or without an offset) throughout the figures to indicate analogous components or features.

FIG. 1A depicts a high-level block diagram of a system for remote health monitoring according to some embodiments disclosed herein.

FIG. 1B depicts another high-level diagram of a system for remote health monitoring according to some embodiments disclosed herein.

FIG. 1C depicts another high-level diagram of a system for remote health monitoring according to some embodiments disclosed herein.

FIG. 1D is a high-level block diagram of procedures for monitoring the health of an individual and providing the individual with guidance and/or coaching messages according to some embodiments disclosed herein.

FIG. 2 depicts a high-level flow diagram of digital circuitry that can be included in a health monitor according to embodiments disclosed herein.

FIG. 3 is a flow diagram of example procedures that can be executed in a health monitor according to embodiments disclosed herein.

FIG. 4 is an illustrative example of a visual interface of a health monitor according to embodiments disclosed herein.

### DETAILED DESCRIPTION

Chronic diseases often affect physical activity of a person. Among elderly, the progression of a chronic disease can be slow, and early symptoms of a chronic disease can go unnoticed. Further, since the senior citizen population often desires to live independently, among senior citizens and the elderly there is often a higher risk of delayed detection of symptoms and conditions that can lead to severe health problems. Therefore, there is a growing need for assistive care technologies, such as remote health monitoring systems that are configured to alert caregivers and other authorized parties with notifications regarding the health status of such individuals (e.g., the elderly and senior citizens). In addition to the elderly and senior citizen population, such systems can be used to monitor health status of other subjects, such as children or generally anyone having a chronic disease.

For example, studies have shown that, in the United States, nearly 13 million community-dwelling older adults live alone. However, maintaining the safety and health of these individuals can be a challenge for caregivers and loved ones, who cannot always be present to tend to these elderly individuals. Therefore, many frail senior citizens can be living alone on many days, during which time major changes in their behavior and health can go unnoticed. This can result in serious health complications and prompt emergency room visits.

As noted above, current solutions in the market often rely on medical alert systems that have very limited capabilities. Although wearable technology (e.g., smart watches capable of detecting a fall) and telehealth application software have been developed, many of such available schemes address a specific health aspect and/or are not designed for the level of digital literacy of many older individuals, many of whom can be considerably less confident using such application software or electronic devices.

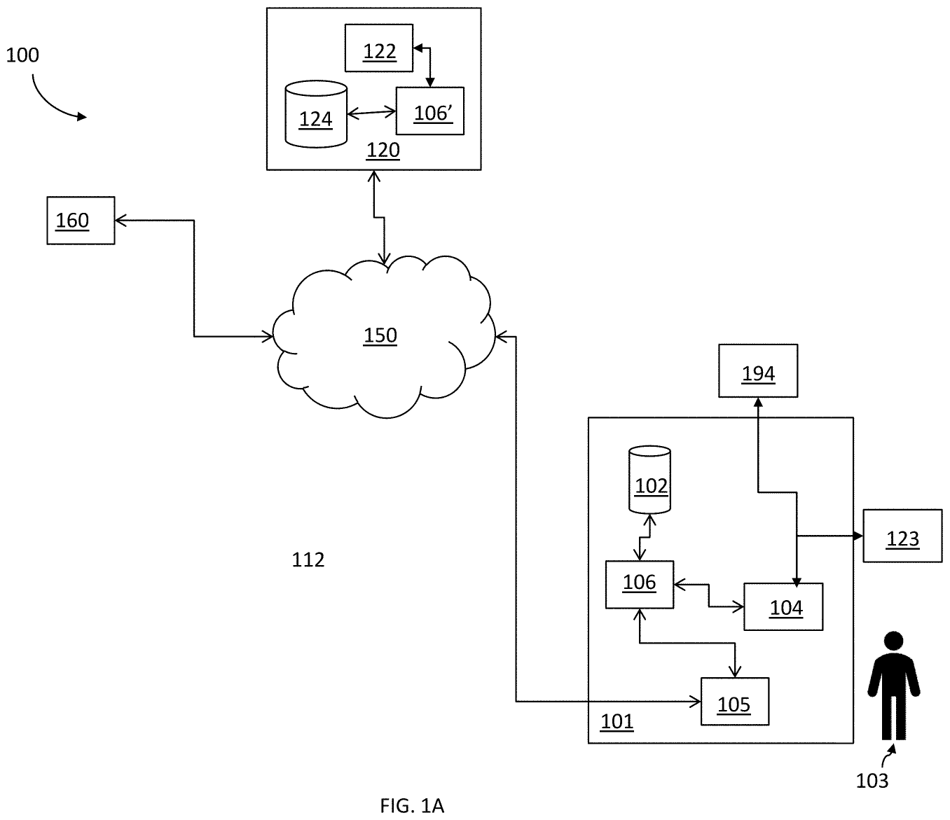

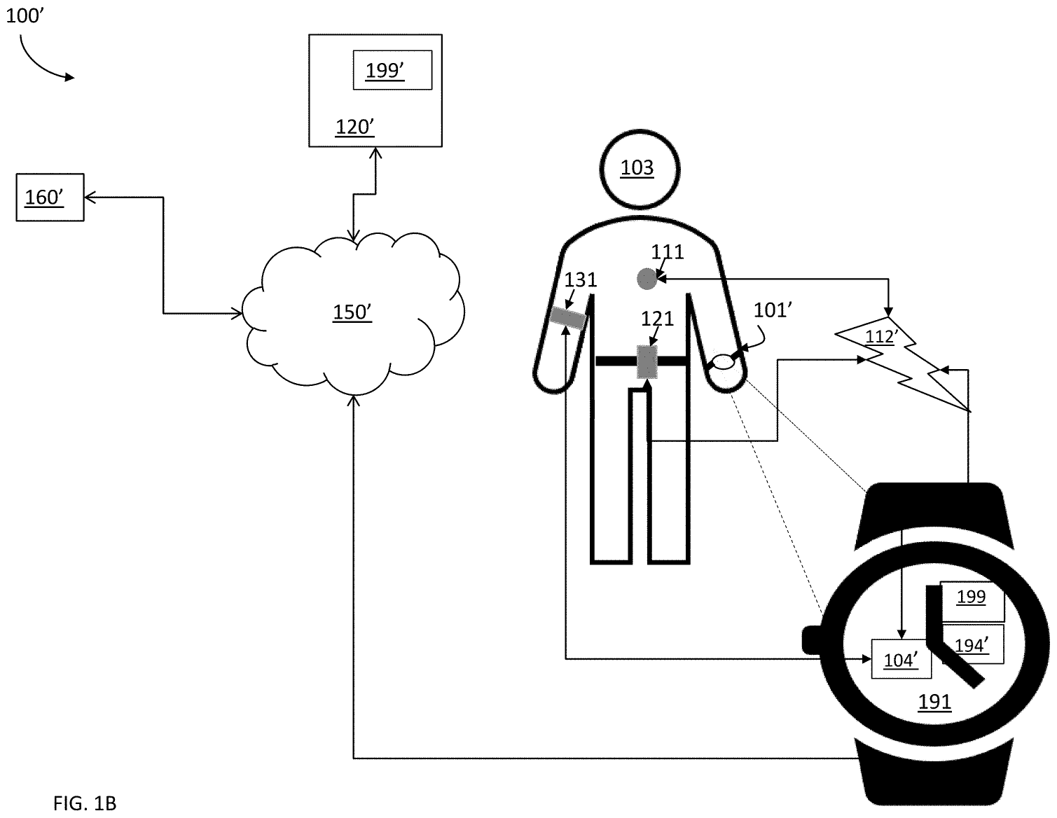

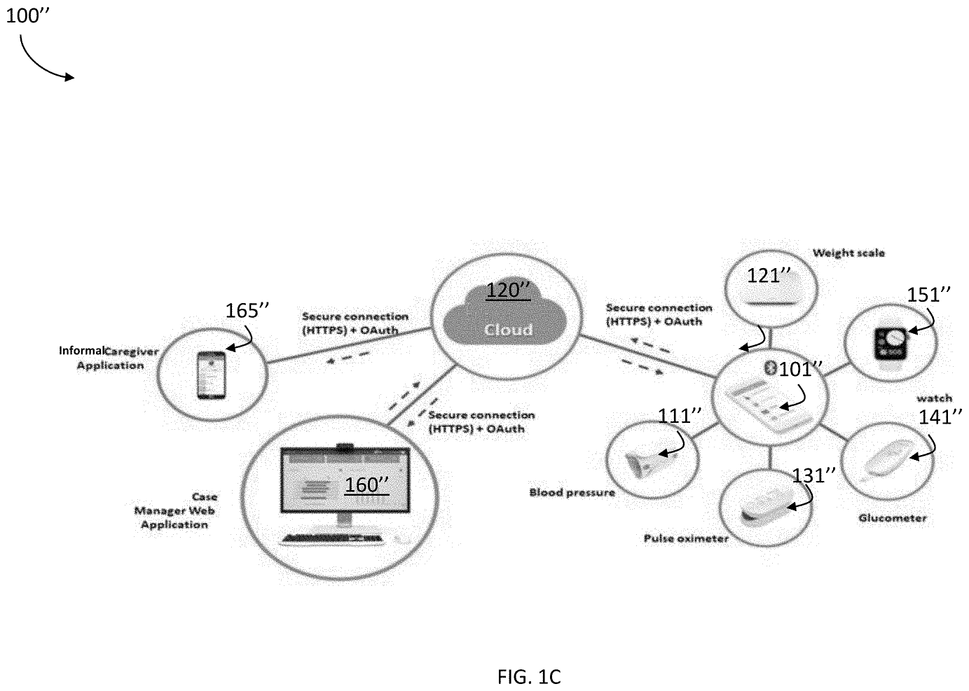

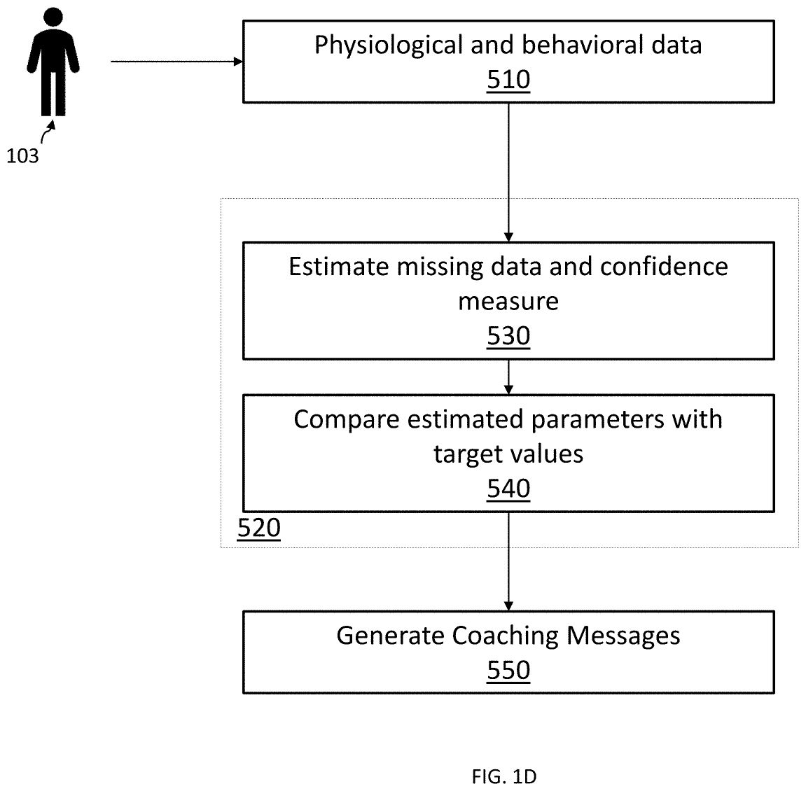

FIG. 1A-1C depict high-level block diagrams of a systems 100/100′/100″ for remote health monitoring according to some embodiments disclosed herein. The systems 100/100′/100″ according to embodiments disclosed herein can provide an unobtrusive remote care monitoring that covers different aspects of individual's health. Further, such systems 100/100′/100″ can be configured to provide early notifications to subjects being monitored, caregivers of such individuals (e.g., senior citizens and people with chronic diseases) about deterioration and possible onset of conditions that require caregiver intervention and/or prevention of possible future complications. Further, some embodiments disclosed herein can report the information gathered from a monitored individual to a third party observer (hereinafter “caregiver” or “case manager”) that continuously monitors the health status of the individual and provides the individual with feedback. For example, embodiments disclosed herein can be configured to monitor the individual's physiological parameters (e.g., sleep, activity, and other parameters whose change can be indicators of health problems) and notify the case manager if a meaningful change in these parameters is detected. The case manager, upon being notified, can evaluate the situation and intervene, if needed, according to preset protocols Further, in some embodiments, the system 100/100′/100″ can generate and send automatic personalized health coaching messages to the individual based on the physiological parameters and behavioral measurements.

Embodiments disclosed herein can also assist individuals and their caregivers in managing chronic diseases. Many older adults (e.g., in some studies about 80%) suffer from at least one chronic disease, while many others have been shown to have at least two chronic diseases (e.g., some studies show about 68% of adults can have at least two chronic diseases). Generally, chronic diseases, if not controlled, can lead to a person's loss of physical functions and independence. Effective self-management of such chronic conditions and diseases can be an important factor in healthy aging, and many such chronic diseases can be alleviated by simple lifestyle changes. Embodiments disclosed herein can provide telehealth assistance to older adults and provide them with information and tools for use in self-management of chronic conditions, and also encourage these individuals to have a more active role in management and treatment of their condition(s).

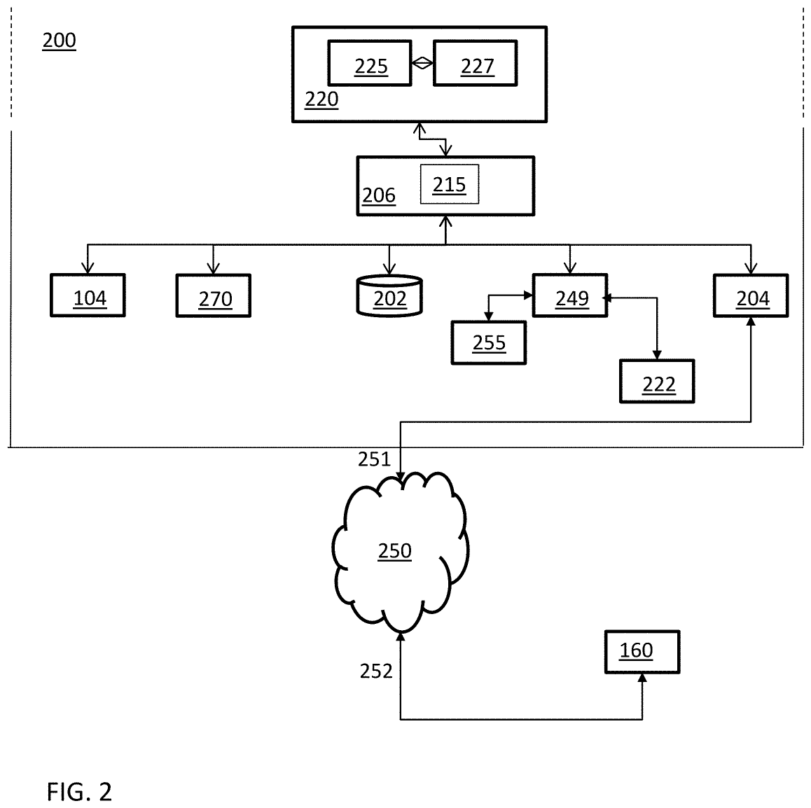

Referring to FIG. 1A, a system 100 according to embodiments disclosed herein can comprise a health monitor 101 having at least one interface 104 configured to receive and collect information relating to physiological condition of the subject 103. The health monitor 101 can also include a processor 106 that is configured to carry out the functions of the health monitor 101. As detailed with respect to FIG. 2, the processor 106 can be any suitable processor known and available in the art.

The health monitor 101 can be implemented in any suitable platform capable of collecting information from the subject, for example in a wearable device, such as a device configured for being worn around the wrist of the subject (e.g., implemented as a watch or in a watch). Further, the health monitor 101 can be implemented in hardware (e.g., in a chip) and/or software stored in processing digital circuitry of an electronic device. For example, the health monitor 101 can be implemented as a chip included in a wearable device (e.g., a watch or a wristband) or be implemented in processing digital circuitry of an electronic device (e.g., a mobile communications device (e.g., phone or a smart watch) carried or worn by the subject).

In some embodiments, the health monitor 101 can be implemented in an existing wearable device, such as a smart watch. Such wearables can be suitable for long term and continuous monitoring of physiological and behavioral parameters along with the geographical information. Such devices are often easily available to public and used by many people and, as such, can be expected to be used by a subject on a regular basis. Alternatively or additionally, the health monitor disclosed herein 101 can be integrated with device, such as a smart phone, that many people often carry. Therefore, a Smartphones incorporating a health monitor 101 according to embodiments disclosed herein can provide a great platform for patient awareness and collaborative caregiving by including family and caregivers in a care circle.

Referring back to FIG. 1A, the interface 104 can be configured to couple the health monitor 101 to one or more sensors 194, each configured to collect and/or obtain some information regarding at least some feature of the subject's health and/or physiological condition. The interface 104 can communicate with the one or more sensors through any communications means and protocol known in the art. For example, the interface 105 can comprise Bluetooth® capabilities and be configured such that it can allow the health monitor 101 to communicate with other devices or portions of the systems (e.g., other wearables, sensors, etc.) via a Bluetooth® connection. Additionally or alternatively, the one or more sensors 194 can be included in the wearable and directly coupled to the interface 104 and/or the processor 106 of the health monitor 101. Specifically, the one or more sensors 194 can be an integral part of the health monitor 101 (e.g., built into the health monitor) or positioned remote from the health monitor 101 and configured to communicate with the health monitor 101 via the interface 104.

The one or more sensors 194 can comprise any sensors known in the art, for example, a positioning sensor (e.g., global positioning sensor (GPS)) for sensing the location of the subject, an accelerometer for detecting the subject's activities, a gyroscope for detecting the body posture of the subject 103, a heart rate detector, such as a red and infrared LED and/or a photodiode for heart rate measurement and/or reflective plethysmography to measure the blood oxygenation, an altimeter and/or a pressure sensor, an audio sensor, and/or a temperature sensor. As noted, the one or more sensors 194 can be locally disposed on the health monitor 101 and/or be positioned remote from the health monitor 101 and configured to communicate with the health monitor 101 via a communications link 112.

The information collected by the one or more sensors 104 can be stored locally (e.g., in a database 102 of the health monitor 101, described in more details below) and processed by the processor 106 and/or be transmitted via a communications network 150 to a server 120 that is configured to monitor, process, and/or store such information.

The processor 106 can be configured to collect and process the physiological parameters obtained by the one or more sensors 194, and process the collected information to detect possible outliers and inaccurate measurements due to improper measurement or motion artifacts. For example, the one or more sensors 194 can include a scale that obtain daily measurements of a subject's weight. The processor 106 can receive and collect the daily weight measurements and detect outliers (e.g., a sudden significant weight loss or weight gain such as a weight loss or gain of more than 10 pounds from one day to next) and record such measurements as being due to improper measurements and/or artifacts.

The processor 106 can further conduct and complete various functions of the health monitor 101. For example, as described in more details below, the processor 106 of the health monitor 101 can process the activity of the person/subject 103 along with physiological parameters, obtained from the one or more sensors 194, to detect possible health issues. For example, after an activity such as climbing the stairs, an abnormal increase of the heart rate or abnormal decrease in the oxygen saturation can be interpreted by the processor 106 as a sign of a potential health issue, and tracked as an important event.

Further, the health monitor 101 can include a communications interface 105 that provides the health monitor 101 with the required capability for establishing a connection to the communications network 150 and the server 120. The communications interface 105 can provide the health monitor 101 with capabilities for connecting to the communications network 150 and the server 120 using any technology or scheme known in the art. For example, the interface 105 can comprise Bluetooth® capabilities and be configured such that it can allow the health monitor 101 to communicate with other devices or portions of the systems (e.g., other wearables, sensors, etc.) via a Bluetooth® connection. Additionally or alternatively, the health monitor 101 can comprise a communication interface 105 that provides the health monitor 101 with capability for establishing a Wi-Fi connection to connect to the server 120 via a communications network 150 (e.g., Internet). Further, the communications interface 105 can be configured to provide cellular communications to provide the health monitor 101 with a connection to the communications network 105 (e.g., the Internet) to carry data and/or voice. Generally, as noted above, any suitable communications medium or technique (e.g., via a wireless or wired connection) can be used with the embodiments disclosed herein to connect the health monitor 101 to the communications network 150 and the server 120. Further, the communications network 150 can generally be any suitable communications network known in the art. For example, the communications network can be the Internet.

The server 120 can be configured to receive and process the information and data collected by the interface 104 using the one or more sensors 194. Specifically, the server 120 can comprise a processor 106′ that receives and processes the data forwarded by the health monitor 101. It should be noted that processing of the data collected by the sensor can occur one the health monitor side (e.g., by processor 106) or on the server side (e.g., by processor 106′). Generally, embodiments disclosed herein can process and analyze any part or portion of the data at any suitable location, on the health monitor 101 and/or on the server 120.

As explained in further details below, embodiments disclosed herein can process and analyze the information and, based on the processed information, generate alarms, notifications, and updates regarding the subject 103. Such alarms, notifications, and updates can include updates regarding events that may be of interest to a caregiver 160 or an entity monitoring the health of the individual 103. For example, upon detecting a fall in an elderly subject 103, the server 120 can generate an alarm signal notifying the subject's 103 caregivers, physician, or any entity 160 that can provide the subject with assistance.

In some embodiments, the monitoring services of the server 120 can be offered as a subscription-based service. For example, as shown in FIG. 1B, some embodiments disclosed herein can offer the monitoring services of the server 120/120′ as a subscription-based service (e.g., provided using a telemonitoring system) in order to assist older adults with monitoring services while they live independently in their own homes. As explained in more details below, embodiments disclosed herein can unobtrusively monitor physiological and behavioral parameters that describe the health conditions of the monitored individuals in near real-time.

As shown in FIG. 1B, the subject 103 can be coupled to a health monitor 101′. As noted above, the health monitor 101′ can be implemented in any suitable medium. For example, in the example shown in FIG. 1B, the health monitor 101′ is displayed as having been implemented in a wrist band or watch 191.

As detailed above, The health monitor 101′ can configured to communicate with one or more sensors 194′, 111, 121, 131, through an interface 104′. As shown, the one or more sensors 194′ can be integrally incorporated in the health monitor 101′ and/or be disposed remote from the health monitor and configured to communicate with/connect to the health monitor 101′ via one or more communications links 112′. As noted above, the communications links 102′ can be any suitable communications link, for example wireless or wired communications links (e.g., a Bluetooth® connection).

Further, the one or more sensors or interfaces 111, 121, 131 can be any suitable sensors or wearables. For example, the one or more additional sensors 111, 121, 131 can be physiological measurement systems that are configured to collect physiological parameters such as blood pressure, blood glucose, weight, respiratory flow and etc. In the example shown in FIG. 1B, the subject 103 is shown as having been coupled with a number of sensors including a wearable ECG patch 111, a pedometer 121, a blood pressure monitor 131, and/or any other suitable wearable sensor or interface. However, any suitable sensor and/or interface can be used to obtain, monitor, and track the subject's health data. These sensor transmit their collected data to the health monitor 101′ via the communications link 112′ (e.g., for sensors 111, 121, 131 that are placed remote from the health monitor 101′) and/or through direct communication with the health monitor (e.g., for sensors directly incorporated within the health monitor 101′ (e.g., one or more sensors 194′)).

The health monitor 101′ can also receive measurements obtained directly by the subject 103 and/or the caregiver. For example, a subject 103 and/or caregiver can obtain some information regarding the subject's physiological and/or health state and input this information and directly provide this information to the health monitor 101′ and/or the server 120′. For example, in some embodiments, at least one of the health monitor 101′ and/or the server 120′ can include a user interface 199/199′ that is configured to receive this information from the subject 103 and/or the caregiver. For example, the interface can be a software interface configured for display on a display of a digital communications device (e.g., computer or smart phone) that is configured to receive the information input by the subject and/or the caregiver.

In some embodiments, the health monitor 101′ can also connect to smart medication boxes or other medication management devices (not shown) and collect information about person's medication adherence.

Generally, the information and parameters collected by the interface 104′ can comprise any suitable information that can be useful in assessing and tracking the subject's physiological condition. For example, the one or more interfaces can collect information regarding at least one of the subject's heart rate, respiration rate, oxygen saturation, location, physical activity level, amount of sedentary behavior, posture and postural transition times, energy expenditure, occurrence of falls, gait and balance, tremor, medication adherence, and sleep patterns.

The server 120′ can process the information collected from the various sensors and interfaces and interpret the information in order to determine possible health issues, and in the event a possible health issue is detected, report the issue to a caregiver 160′. Specifically, the server 120′ of the health monitor 101′ can receive and process information regarding the activity levels of the person/subject 103 along with various physiological parameters, obtained from the one or more sensors 194′, 111, 121, 131 to detect possible health issues. For example, after an activity such as climbing the stairs, an abnormal increase of the heart rate or abnormal decrease in the oxygen saturation can be interpreted by the processor 106′ as a sign of a potential health issue, and tracked as an important event.

It should be noted that although described as conducting the processing and analysis of data at the server 120′ side, processing of any portion of the information as described in relation to embodiments disclosed herein can occur at the health monitor 101′ and/or at the server side 120′.

Regardless of where the processing of the collected data is carried out (at the server 120′ and/or the health monitor 101′), embodiments disclosed herein can employ the processed data to detect significant/meaningful changes in the patterns of sleep, activity and physiological parameters that can be early indications of health problems in older adults, and notify a caregiver 160′.

The caregiver 160′ in some subscription service embodiments can be an entity (e.g., individual or a facility) that is familiar with the subject's history and has access to her/his medical data. The caregiver 160′ can evaluate the information provided by the server (e.g., the processed data) and intervenes according to the protocols developed by health professionals. The subscription based service can also coordinate with informal caregivers (e.g., relatives of an elderly individual) about important events and update them about the status of the older adult's health through a mobile application designed for such informal caregivers.

FIG. 1C is another high-level diagram of a system 100″ for remote health monitoring according to some embodiments disclosed herein. As shown, the health monitor 101″ can be implemented on a personal communications device (e.g., smart phone) carried by the subject. The health monitor 101″ can be implemented in hardware and/or as computer readable instructions that are stored in the memory of the digital circuitry of the communications device. The health monitor 101″ can be configured to be coupled to a number of devices, sensors, and interfaces and receive information regarding the health and physiological condition of the subject from these components. For example, as shown in FIG. 1C, the health monitor 101″ can be configured for coupling to a blood pressure 111″, a smart weight scale 121″, a pulse oximeter 131″, a glucometer 141″, and a smart watch 151″, and receive information and data from these components.

The information obtained by the various sensors, devices, and interfaces can comprise physiological information, such as heart rate, blood pressure, blood glucose, oxygen saturation. The behavioral parameters can include daily and hourly steps walked, active energy, exercise time and sleep time. For example, a smart watch 151″ can be used to track information such as daily steps walked, sleep hours, and exercise time.

Based on the collected information, an individualized probabilistic model of behavioral parameter can be created from the historic data obtained from each subject. Specifically, data collected from each subject over a predetermined period of time (e.g., over a day, over a 24-hour period, over a week, etc.) can be fitted and modeled as a suitable statistical distribution, such as a Gaussian distribution. For example, data collected from the number of steps a person takes during a time period (e.g., day or a 24-hour period) can be modeled as a Gaussian distribution. Under such distribution, the number of steps Sᵢ taken by the individual at the i-th hour of a day can be represented as having a mean value of μᵢ and a standard deviation of σᵢ, where the mean μᵢ and standard deviation σᵢ can be estimated from the historic data:

Sᵢ˜N(μᵢ,σᵢ)

If the person is wearing the watch at all times, the number of daily steps S_{d} (over a 24-hour period) can be calculated as:

$${s}_{d} = {\sum }_{i = 0}^{24} {S}_{i}$$

However, many individuals cannot be expected to wear their wearables (e.g., smart watch) at all times. Therefore, it can be expected that some individual being monitored does not wear his/her at least during some periods of time over the span of a day. Embodiments disclosed herein compensate for the missing information for by estimating the total daily steps taken by such individuals using the number of steps taken at known intervals plus the mean value of the unknown time intervals. Therefore, the estimated daily step count [symbol] can be represented as:

$$= {\sum }_{known} {S}_{i} + {\sum }_{unknown} {μ}_{j}$$

A confidence interval CI for the estimated daily step count [symbol] can be calculated as:

$$CI = {\sum }_{unknown} {σ}_{i}$$

Further, a confidence measure CM of the daily activity estimation can be calculated as:

$$C M = \frac{- CI}{+ CI}$$

The confidence value can have a value between 0 and 1, where the confidence value of 1 demonstrates the highest level of confidence and the confidence value of 0 demonstrates the lowest level of confidence.

Using these metrics, embodiments disclosed herein can predict a person's behavior using information and health data previously obtained from the person. In other words, information regarding physical activity and/or sleep pattern of a monitored individual can be derived, using a probabilistic model (e.g., any suitable statistical distribution, such as a Gaussian distribution) from the historical data that indicates the probability that the individual is asleep, awake, having restful sleep, having interrupted sleep, moving and engaged in a physical activity, or is stationary at any given time during a 24-hour period. Similarly, using the probabilistic model, based on the historical data, embodiments disclosed herein can predict the number of times that the person has had interrupted sleep during the night. Further, the probabilistic model can be used to predict sleep duration, time the person is expected to go to sleep, and the number of times the person has been interrupted during the sleep.

Accordingly, embodiments disclosed herein can apply this probabilistic model to historical data in order to predict and estimate a person's behavior during time intervals (“missing time intervals”) that the person is not carrying/wearing the health monitor 101″.

The confidence interval for each estimated event can then be calculated. Specifically, using the probabilistic model, the expected value and the confidence interval of behavioral parameters (e.g., sleep duration or amount of activity) can be estimated from the probabilistic model for the missing intervals. An estimated value for the total duration of the sleep or amount of activity within the 24 hour period can then be calculated from the known information and the probabilistic model and a confidence interval is generated for each estimation.

Similarly, when using blood pressure measurements 111″, a confidence value can be calculated based on the historic values and possible physiological range of the systolic and diastolic values. When a blood pressure measurement with low confidence (e.g., when the reported systolic value is very close to the diastolic value (which is not physiologically possible) and/or the systolic and diastolic values are outside the expected range based on the average and standard deviation of one week of blood pressure values) is received, a message can be sent to the subject to request another measurement along with instruction for correct measurement including a five minute rest time and no movement during measurement. A weekly average of blood pressure values can be calculated and compared to the historic values and age specific target values. Changes in the average blood pressure can be used to inform the user about their blood pressure and outside target values will trigger reminder messages for medication adherence. For example, in one embodiment, when monitoring a person who is controlling his/her hypertension by regularly taking medication, if a blood pressure reading outside (e.g., outside of a set threshold, outside of a normal range) of the historic daily estimate is detected, the system 100″' can generate and transmit a notification and/or a reminder to the individual regarding taking his/her medications.

The collected data from users can be stored on an SSL secured database and various level of access to the data of the users can be managed. The processed and collected physiological and behavioral information along with important detected events can be transferred to the server 120″ (e.g., a HIPAA compliant remote cloud server 120″) in near real time through a secured Internet connection. In some embodiments, the collected information on the server 120″ or cloud server can be processed and stored depending on the caregiver preference and the subject's medical condition.

The cloud server 120″ can include any suitable information for user authentication (e.g., several RESTful API's), receiving data in various formats from the user's mobile application, and sending remote notifications and responding to queries from case manager web dashboard and Insight Caregiver mobile application.

Upon completion of processing by the cloud server 120″, the processed information can be forwarded to a caregiver, who may be the case manager assigned by a healthcare provider to the subject being monitored. In some embodiments disclosed herein, the case manager can be coupled with a web-based dashboard (e.g., a web-based application) that is configured to allow the case manager to access to subject's health data, including recent data in near real-time and daily and weekly trends.

The server 120″ can be configured to update this health data as it receives new/additional information from the subject (e.g., over predetermined time periods, for example hourly or daily) so that the case manager can access the latest events and data in near real-time. The server 120″ can also provide informal caregivers 165 with latest update regarding the subject's health.

In some embodiments, the caregiver and/or the informal caregiver can also acquire the latest information about the subject's health status and latest physiological measurements. Various notifications can also be set on health monitor 101″ based on the condition of the person that is monitored. For example, for a patient with a cardiac disease, heart notifications can be set to notify the caregiver about heart rate than may be lower or higher than normal or irregular heart rhythm.

The system 100/100′/100″ can also facilitate and simplify capturing patient generated health data by providing a platform that optimizes coordinated and collaborative caregiving by including family members and caregivers in a circle of care. Specifically, the system 100/100′/100″ can collect physiological and behavioral data with geographical information in real-time. In some embodiments, the data collected by the system 100/100′/100″ can be de-identified to remove any association with the subject(s) from whom the data have been collected. The de-identified data can be stored on the database 124 of the sever 120 and used by various entities, for example for determining possible outbreaks in communities by public health officials. The system 100/100′/100″ can also provide individuals and their caregivers with aware of the individual's health status and proper intervention by the caregivers can prevent more severe illnesses.

Further, the data obtained by the health monitor 101/101′/101″ can comprise continuous and long term physiological data that can be used to accurately and quickly diagnose patients who have been exposed to pathogens. For example, the health monitor 101/101′/101″ can monitor the temperature of residents at a nursing home and, upon detecting a surge in temperature in a predetermined population of the residents (e.g., more than 20%), report to public health officials that there is a possibility of a disease outbreak in the nursing home.

Embodiments disclosed herein can also assist in monitoring various health conditions of the subject. For example, some embodiments disclosed herein can be used in monitoring hypertension. Although conditions such as hypertension can be treated by taking antihypertensive drug therapy, studies show that only a small percentage of older adult (e.g., about 33%˜46%) control their hypertension through antihypertensive drug therapy. However, uncontrolled hypertension can often lead to heart disease, stroke, eye problems, and kidney failure, and often times, the such poor outcomes are results of lack of self-monitoring and sedentary behavior and life style. Self-monitoring in conjunction with medication adherence, education and lifestyle counselling can lead to clinically significant blood pressure reduction, and there is substantial evidence that regular monitoring, routine physical activity, good medication compliance, and a healthy diet can be important factors in reducing the morbidity associated with hypertension.

Additionally, in some embodiment, the processor 106/106′ can be configured calculate a health score in real time for each person based on the subject's age, weight, height, activity, mental status, and physiological parameters. For example, the health score can have a value ranging between 0 to 100, where having a higher value of health score can indicate that the subject is in a better health status than those having lower scores. The trend of this value over time can indicate deterioration of the subject's health (if the score decreases) or improvement of the subject's health status (if the score increases). This score can be used as a marker for the effectiveness of a treatment or side effects of a medication.

The information transmitted to the caregiver can also include the wearable's (i.e., wearable health monitor 101) status in terms of the battery life time, connection to the communication network 150 (e.g., Internet) and whether they are worn by the subject, and if not worn, the last time it was worn.

Additionally or alternatively, the information provided to the caregiver can include notifications regarding parameters, such as the distance of the subject has traveled from home, the amount of time the subject has been outside home, sleep pattern and number of interrupts during sleep, low battery of the wearable, low activity and high sedentary time during the day.

Further, on the remote server 120, short term and long term physiological and behavioral parameters of the person can be processed according to the person's own data and the data of other people with similar health conditions. Significant variation of the statistical properties of those parameters can be detected as possible health issues and the assigned caregiver can be notified about the potential health condition.

In some embodiments, the remote server 120 can also be configured to analyze a correlation, if any, between the medications taken by the person with the physiological parameters and inform the caregiver about the effectiveness of the medications. Further, the tele-monitoring system can be configured so that multiple caregivers or relatives can receive status updates about a single subject or one caregiver can receive status updates about multiple subjects.

Further, in some embodiments, the health monitor 101 can comprise a voice enabled assistant 123 (integrated on the health monitor 101 or a separate system and coupled to the health monitor 101 via the interface 104) that is configured to communicate verbally with the subject. This verbal communication can include reminders for medications, messages that encourage more activity, and/or messages that verify that the subject has taken his/her medication(s). The voice enabled assistant can also be configured to communicate with the subject to ask questions about the mood, level of pain and other possible symptoms of the person, and collect this information for better health assessment. In some embodiments, the verbal communication can include puzzles or mind games that are configured to quantify the mental status of the subject.

FIG. 1D is a high-level block diagram of procedures for monitoring the health of an individual and providing the individual with guidance and/or coaching messages according to some embodiments disclosed herein. As shown with respect to FIG. 1A-1C, the health monitor 101/101′/101″ is configured to receive physiological and behavioral information and data (FIG. 1D, box 510) from a subject 103 being monitored by the system. The physiological and behavioral information and data can be collected from monitoring physiological and behavioral parameters of the subject, such as the subject's pulse, blood pressure, glucose level, amount of physical activity, and sleep patterns. The system collects and stores this data over a period of time and employs this historic data to form an individualized probabilistic model of behavioral and physiological parameters for each subject (box 520).

However, the collected data can be susceptible to noise due to motion and improper use of the wearable devices (e.g., the health monitor or any of its connected sensors and interfaces). Further, since there is a possibility that the subject may not always carry or wear the health monitor, the possibility of having periods of time when no data were collected exists. Embodiments disclosed herein compensate for these missing intervals by estimating missing data and parameters (e.g., data relating to usage of the health monitor, daily steps walked, daily exercise time, sleep duration, recent value and weekly average of blood pressure and other physiological parameters that are needed to monitor a person's overall health) from the collected raw data (box 530).

A confidence interval for each estimated parameter is then generated to be used as a reliability measure in evaluating the information gathered by the health monitor (box 540). For example, in order to estimate parameters such as daily steps walked, sleep hours, and exercise time, an estimation of the missing data is required. For this purpose, historic data is used to develop an individualized probabilistic model of behavioral parameters. The daily behavioral parameters are estimated from the known intervals of data and the probabilistic model. A confidence interval for each estimation is calculated to illustrate the reliability of the estimated parameters. The estimated parameters are then compared to target parameters that are often provided by the subject and/or caregiver (e.g., target sleep time of eight hours/night or target body temperature below 100.4 F). In the event the estimated values fall outside of a target range for that parameter (e.g., sleep duration of between six to eight hours), an alarm is generated by the system to notify the caregiver to provide appropriate intervention.

The confidence measure (CM) can used as a reliability factor for the estimated parameter and used to generate an alarm. For example, if the CM is closer to 1 (e.g., CM≥0.6 or CM≥0.5), there is a high confidence in the estimated parameter and therefore the alarm is allowed to be sent. Similarly, if the confidence measure is below a certain threshold (e.g., CM≤0.4, CM<0.5, or, CM<0.6), the alarm message is not generated. Embodiments disclosed herein can interpret having a low confidence measure as an indication that the available data is not sufficient to generate a good estimate and transmit a message to the user requesting that he/she wears/carries the health monitor more frequently.

In some embodiments, in addition to or in place of direct intervention by a caregiver, automatic personalized health coaching messages can be transmitted to the subject. Specifically, based on the physiological parameters and behavioral measurements collected and estimated by the system, the system can send appropriate automatic personalized health coaching messages to the subject (box 550). The near-real time analysis of the behavioral data can lead to an effective automatic coaching and intervention system that allows for management of chronic diseases, such as hypertension.

In some embodiments, the subject or a caregiver (hereinafter “user”) can request the coaching preferences and request that the system estimate any missing intervals and parameters. Such requests can be made, for example, through the user interface of the health monitor. The user can also request and set the parameters being monitored (e.g., target daily steps walked, exercise time, and sleep hours) and/or their target values. In such embodiments, motivational values can be generated based on comparison of the estimated values and target values of such parameters.

For example, for hypertension patients, average blood pressure values can be compared to historic values and age specific target values, and informational messages regarding the blood pressure control can be generated. The changes in the average blood pressure values can be used as a surrogate for medication adherence and reminders about medication can also be generated. Further, occasional informational and motivational messages regarding heart healthy diet and risky habits, such as smoking and drinking, can also be generated by the system. Additionally or alternatively, in some embodiments, a message queue can be created and stored in the server 120. The messages in the queue can be prioritized to be sent to the user according to the individual's preference regarding the time and frequency of messages. Specifically, since many users may not be interested in receiving multiple messages and notifications during the course of a day, the system can assign a priority to each message, place the messages in a massaging queue based on their priority, and transmit the higher priority messages to the subject before the lower priority messages. For example, messages triggered by a system alarm (e.g., a message alerting a person to take their hypertension medication in response to observation of an abnormal/out of range blood pressure reading) can get priority over messages general health coaching messages (e.g., messages highlighting the downsides of smoking and drinking alcohol).

In some embodiments, in response to determining that intervention is required (e.g., based on comparison of the estimated health data to the target data), the system can provide the subject with health coaching intervention through mobile messaging. Such coaching messages can have a positive impact on physical and mental health of subject. For example, physical activity and Sleep duration have been shown to be important in controlling a person's blood pressure (e.g., hypertension control). Therefore, embodiments disclosed herein can generate messages for providing to individuals requiring blood pressure monitoring that include motivational messages to encourage elements including but not limited to: 1) regular use of the health monitor, 2) regular blood pressure measurements, 3) healthy lifestyle regarding sleep duration and activity, 4) medication adherence, 5) adherence with their AHA recommended heart-healthy diet, and 6) reducing smoking, caffeine and alcohol consumption. Data regarding items 1-3 can also be used to personalize health coaching messages. For other items, educational and motivational messages can be provided to the subject.

FIG. 2 depicts a high-level illustration of some of the digital circuitry that can be included in the health monitor 101. Generally, the health monitor 101 can comprise various digital electronic circuitry or computer hardware 200 that can be used with, incorporated in, or fully or partially included in the health monitor 101 according to the embodiments disclosed herein. As shown, the electric circuitry 200 can include a processor 206 that is configured to monitor the operation of the health monitor 101, send and/or receive signals regarding the operation of the health monitor 101, and/or control the operation of the health monitor 101.

The processor 206 can be configured to collect or receive information and data regarding the operation of the health monitor 101 and/or store or forward information and data to another entity (e.g., care provider 160 or server 120, etc.). The processor 206 can further be configured to control, monitor, and/or carry out various functions needed for analysis, interpretation, tracking, and reporting of information and data collected by health monitor 101. Generally, these functions can be carried out and implemented by any suitable computer system and/or in digital circuitry or computer hardware, and the processor 206 can implement and/or control the various functions and methods described herein. The processor 206 can further be generally configured to monitor the operation of the health monitor 101, send and/or receive signals regarding the operation of the system 200, and/or control the operation of the system 200.

For example, the processor 206 can be coupled to the one or more interfaces 204 of the health monitor 101 and configured to receive information regarding the physiological condition of the subject from the health monitor 101. The processor 206 can process this information to perform various functions, such as determine a health score for the subject as described above. The processor can be configured to execute instructions to perform one or more tasks in response to receiving information from the one or more interfaces 204. For example, the processor 206 can be configured to execute instructions configured to quantify severity of any undesired event (e.g., falls) detected by the interface(s) 204. In some embodiments, the processor 206 can be configured to quantify the severity of the undesired event. For example, the processor 206 can comprise instructions that can quantify undesired events.

The processor 206 can further be configured to generate a notification in response to receiving information (from the interfaces(s) 204) which can be of interest to the authorized party 160 and/or transmit the generated notification to the authorized party 160. The notification can be transmitted to the authorized party using any scheme known and available in the art. For example, the system 200 can be configured to transmit the notification via a communications network 250. The communications network 250 can be any communications network known and available in the art. Further, the system 200 and the processor 206 can use any means (e.g., communications links, communications protocols, etc.) known and available in the art to communicate with the authorized party 160. The system 200 can include any communications means necessary to communicate with the authorized party via the communications network 250.

In some embodiment, the authorized party 160 can be a designated device (e.g., device being used by a case manager, a caregiver, or an informal caregiver) configured to receive the notification 251 generated by the health monitor 101. The designated device can be any suitable device known and available in the art. For example, the designated device can be any of a mobile device, a desktop computer, earbud, smart glasses with pop-up message window.

The third party/designated device 160 can be configured to issue a response signal 252 to the health monitor 101 in response to receiving the notification generated by the health monitor 101. The response signal 252 can comprise instructions that can be executed by the processor 206 to perform one or more tasks. For example, the one or more instructions can comprise instructions that, once executed by the processor, performing at least one or more of issuing an alarm signal to the subject.

As noted, the health monitor 101 can be configured to generate an alarm signal in response to detection of undesired events (e.g., a fall). In some embodiments, the alarm signal generated by the health monitor 101 can be output through at least one speaker 222. The processor 206 can be coupled to the at least one speaker 222 via an input/output (I/O) interface 249 of the health monitor 101 and configured to instruct the speaker 222 to generate an alarm signal if/when an undesired event (e.g., call) is detected. The alarm signal can comprise a message in natural language.

Referring back to FIG. 2, the processor 206 can be connected to a main memory 220, and comprise a central processing unit (CPU) 215 that includes processing circuitry configured to manipulate instructions received from the main memory 220 and execute various instructions. The CPU 215 can be any suitable processing unit known in the art. For example, the CPU 215 can be a general and/or special purpose microprocessor, such as an application-specific instruction set processor, graphics processing unit, physics processing unit, digital signal processor, image processor, coprocessor, floating-point processor, network processor, and/or any other suitable processor that can be used in a digital computing circuitry. Alternatively or additionally, the processor can comprise at least one of a multi-core processor and a front-end processor.

Generally, the processor 206 and the CPU 215 can be configured to receive instructions and data from the main memory 220 (e.g., a read-only memory or a random access memory or both) and execute the instructions. The instructions and other data can be stored in the main memory 220. The processor 206 and the main memory 220 can be included in or supplemented by special purpose logic circuitry. The main memory 220 can be any suitable form of volatile memory, non-volatile memory, semi-volatile memory, or virtual memory included in machine-readable storage devices suitable for embodying data and computer program instructions. For example, the main memory 220 can comprise magnetic disks (e.g., internal or removable disks), magneto-optical disks, one or more of a semiconductor memory device (e.g., EPROM or EEPROM), flash memory, CD-ROM, and/or DVD-ROM disks.

The main memory 220 can comprise an operating system 225 that is configured to implement various operating system functions. For example, the operating system 225 can be responsible for controlling access to various devices, memory management, and/or implementing various functions of the health monitor 101. Generally, the operating system 225 can be any suitable system software that can manage computer hardware and software resources and provide common services for computer programs.

The main memory 220 can also hold application software 227. For example, the main memory 220 and application software 227 can include various computer executable instructions, application software, and data structures, such as computer executable instructions and data structures that implement various aspects of the embodiments described herein. For example, the main memory 220 and application software 227 can include computer executable instructions, application software, and data structures, such as computer executable instructions and data structures that implement the various functions of the health monitor 101, which can be employed to receive and analyze subject's health information from the various sensors and interfaces. Generally, the functions performed by the health monitor 102 can be implemented in digital electronic circuitry or in computer hardware that executes software, firmware, or combinations thereof. The implementation can be as a computer program product (e.g., a computer program tangibly embodied in a non-transitory machine-readable storage device) for execution by or to control the operation of a data processing apparatus (e.g., a computer, a programmable processor, or multiple computers).

The main memory 220 can also be connected to a cache unit (not shown) configured to store copies of the data from the most frequently used main memory 220. The program codes that can be used with the embodiments disclosed herein can be implemented and written in any form of programming language, including compiled or interpreted languages, and can be deployed in any form, including as a stand-alone program or as a component, module, subroutine, or other unit suitable for use in a computing environment. A computer program can be configured to be executed on a computer, or on multiple computers, at one site or distributed across multiple sites and interconnected by a communications network, such as the Internet.

The processor 206 can further be coupled to a database or data storage 202. The data storage 202 can be configured to store information and data relating to various functions and operations of the health monitor 101. For example, the data storage 202 can store the data collected by the health monitor 101. Further, in some embodiments, the database 202 can be configured to store information regarding events that may be of interest to the authorized party 160. For example, the database 202 can be configured to store the number of detected sudden acceleration or deceleration events that can indicate a fall.

The processor 206 can further be coupled to a display 270. The display 270 can be configured to receive information and instructions from the processor. The display 270 can generally be any suitable display available in the art, for example a Liquid Crystal Display (LCD) or a light emitting diode (LED) display. For example, the display 270 can be a smart and/or touch sensitive display that can receive instructions from a user.

The processor 206 can further be connected to various interfaces. The connection to the various interfaces can be established via a system or an input/output (I/O) interface 249 (e.g., Bluetooth, USB connector, audio interface, FireWire, interface for connecting peripheral devices, etc.). The I/O interface 249 can be directly or indirectly connected to the health monitor 101. The processor 206 can further be coupled to a communication interface 204, such as a network interface. The communication interface 204 can be a communication interface (shown FIG. 1A) that is included in the health monitor 101 and/or a remote communication interface 204 that is configured to communicate with the health monitor 101. For example, the communication interface 204 can be a communications interface that is configured to provide the health monitor 101 with a connection to a suitable communications network, such as the Internet. Transmission and reception of data, information, and instructions can occur over the communications network. Further, in some embodiments, the communication interface 204 can be an interface that is configured to allow communication between the digital circuitry (e.g., a remote computer) and the health monitor 101 (e.g., via any suitable communications means such as a wired or wireless communications protocols including WIFI and Bluetooth communications schemes).

As noted, the health monitor 101 can be configured to issue instructions or alarms to the subject and/or the authorized party. In some embodiments, the health monitor 101 can be implemented in the electronic circuitry of the health monitor 101, for example in application software 227, and configured such that one or more instructions and/or alerts can be stored in the form of instructions and/or audio files (e.g., in the form of Waveform Audio File Format) in the main memory 220. The health monitor 101 can be configured such that upon initialization of the health monitor 101, the processor 206 transfers audio files for instructing the subject or issuing alerts from the main memory 220 and causes the execution of the files. The subject instruction system can communicate, via the I/O interface 249, with the one or more speakers 22 of the health monitor 101, and instruct the speakers 222 to play the relevant audio files for the subject.

The verbal communication with the subject can be conducted using natural language. Additionally or alternatively, the verbal communication with the test subject can be performed by using one or more pre-recorded messages configured for delivery to the test subject. The pre-recorded messages can be stored in the database 202 and accessed by the processor 206 as needed. The processor 206 can use the audio speaker 222 to conduct verbal communication with the subject. In some embodiments, the health monitor 101 can include a microphone for receiving commands, instructions, or requests from the subject and/or caregiver.

As noted above, the health monitor 101 can further comprise an interface 255/199 configured to receive information from the subject or the caregiver. The interface 255 can be coupled to the I/O interface 249 of the system 200 such that information received by the interface 255 are directed, through the I/O interface 249 to the processor 206. Similarly, the interface 255 can be configured to receive instructions from the processor 206 through the I/O interface 249.

Additionally or alternatively, the health monitor 101 can include Bluetooth capabilities for communication to other wearable or non-wearable systems, Global Positioning System (GPS) for sensing the location of the subject, one or more accelerometers for detecting the activity of the subject, one or more gyroscopes for detection of body posture of the subject, a red and infrared LED and a photodiode for heart rate measurement and reflective plethysmography to measure the blood oxygenation, an altimeter and pressure sensor, a temperature sensor, and/or a battery charger system. Further, in some embodiments, the health monitor 101 can comprise a rechargeable battery.

Further, the health monitor 101 can be coupled with other wearables. The subject can use other wearables once in a while for a specific time interval. For example a subject with hypertension can wear the blood pressure monitor over predetermined intervals (e.g., six months) for a few days to obtain blood pressure measurements over predetermined time periods (e.g., every hour). Alternatively or additionally, other wearables such as a waist wearable can be used over predetermined intervals (e.g., six months) for a few days for gait and balance assessment of the patient. Similarly, a wearable ECG patch can be used over predetermined intervals (e.g., six months) for assessment of the cardiac rhythms of the patient.

For example, as shown in FIG. 1B, the health monitor 101 can include or be coupled with a wearable patch 111 that is disposed on the subject's body (e.g., the subject's chest). The patch 111 can comprise a chest strap or adhesive having at least one of a processing unit and memory, 1 lead ECG, accelerometer, gyroscope for posture detection, Bluetooth capabilities to communicate with the health monitor 101, a rechargeable battery, and/or a battery charger module.

Further, as also shown in FIG. 1B, the health monitor 101 can include or be coupled with a wearable 121 (e.g., worn around the waist) that can be worn in any suitable manner, for example with a belt. The wearable 121 can comprise a processing unit and memory, an accelerometer, a gyroscope, Bluetooth capabilities for communicating with the health monitor 101, a rechargeable battery, and/or a battery charger module.

Still further, as also shown in FIG. 1B, the health monitor 101 can include or be coupled with a wearable 131 (e.g., worn around the arm) that can comprise a processing unit and memory, an Oscillometric blood pressure measurement module, an inflatable cuff, Bluetooth capabilities for communicating with the health monitor 101, a rechargeable battery, and/or a battery charger module.

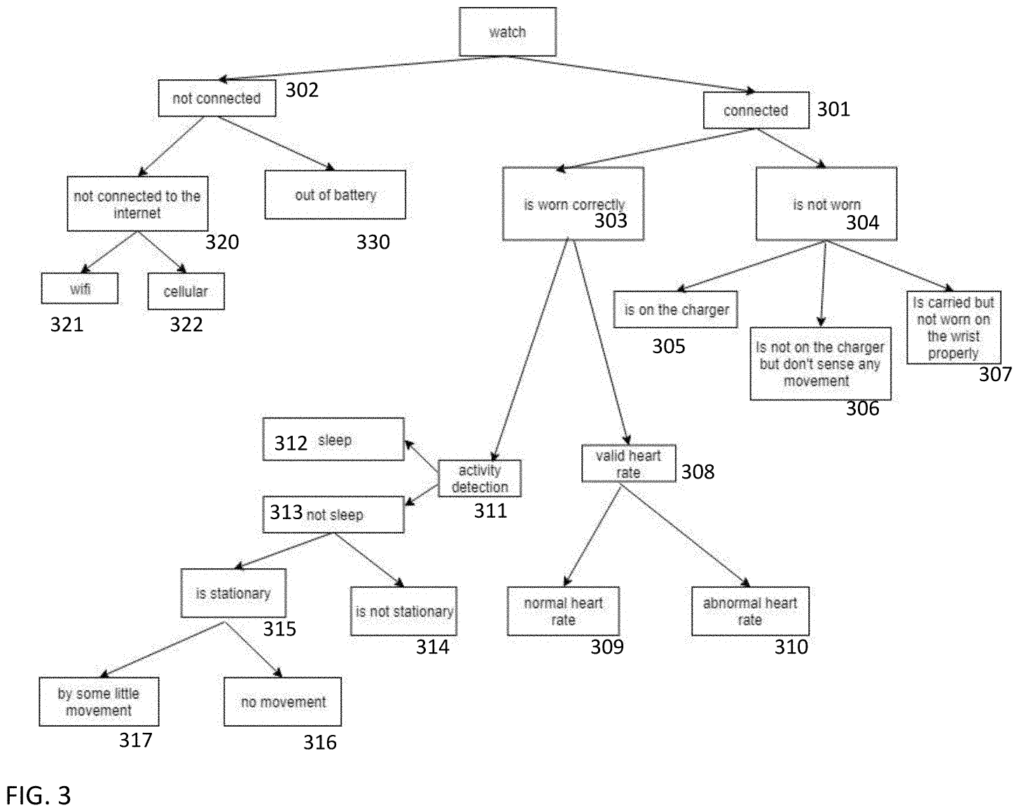

FIG. 3 is a flow diagram of example procedures that can be executed in a health monitor according to embodiments disclosed herein. As shown, the health monitor 101 (e.g., a wearable watch) can determine whether the device is connected 301 or not connected 302. If the device is connected 301, the health monitor can determine whether the device is worn 303 or not worn 304. If the device is not worn 304, the wearable can determine whether the device is on the charger 305, is not on the charger and no movement is sensed 306, or is carried but not worn on the wrist properly 307.

If the device is worn correctly 303, the device can determine whether the subject has a valid heart rate 308. If a valid heart rate is detected 308, the device can determine whether the heart rate is normal 309 or abnormal 310. Lack of heart rate 308 or presence of an abnormal heart rate 310 can trigger an alarm to a care provider.

If the device is worn correctly 303, the device can determine whether the subject is moving 311. Specifically, the device can determine whether the subject is sleeping 312 or not sleeping 313. If the subject is not sleeping 313, the device can determine if the subject is not stationary 314 or stationary 315. If the subject is stationary 315, the device can determine if the subject is moving 317 or if there is no movement 316. Each of these situations can trigger an appropriate alarm or lead to collection of relevant data.

If the device is not connected 302, the health monitor can determine whether it is out of battery 330 and/or whether the device is not connected to the communications network (e.g., Internet) 320. If no network connection is detected 320, the health monitor can attempt to connect to an alternate communications network (e.g., via cellular 322 or Wi-Fi 321).

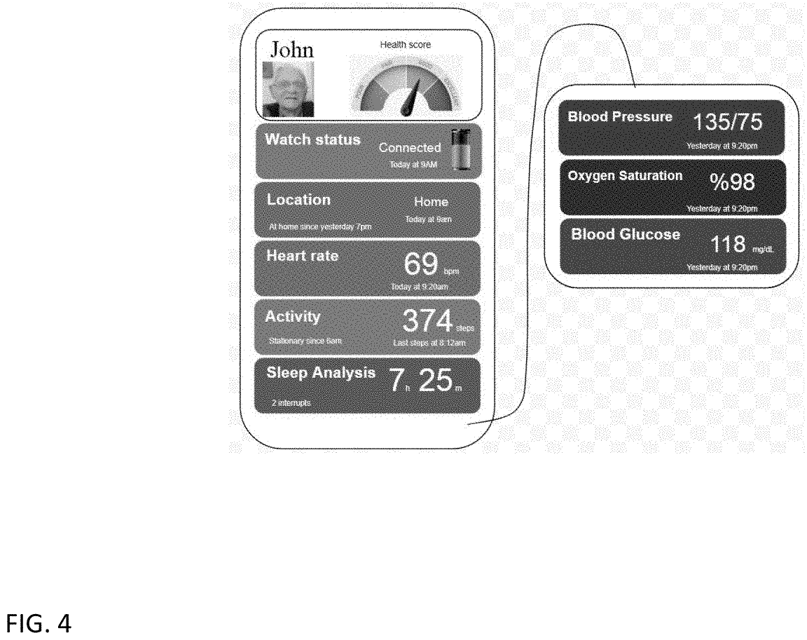

FIG. 4 is an illustrative example of a visual interface of a health monitor according to embodiments disclosed herein. As shown, the health monitor can receive information such as blood pressure, blood oxygen saturation level, and blood glucose level of the subject from connected interfaces, systems, and/or sensors. The visual interface can display (to the caregiver or authorized party and/or to the subject) information such as the status of the wearable (battery level, connectivity, etc.), location of the subject, heart rate of the subject, activity level of the subject.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

What is claimed is:

1. A system comprising:
one or more sensors each configured to detect a first plurality of parameters relating to physiological condition of a subject;
an interface configured to receive a second plurality of parameters relating to the physiological condition of the subject; and
at least one processor coupled to the one or more sensors and the interface, the processor being configured to monitor the first and second pluralities of parameters and, in an event at least one parameter falls outside of a predetermined range, issue an alarm signal indicating onset of an undesired event in the physiological condition of the subject.

2. The system of claim 1, wherein the one or more sensors comprise at least one of a heart rate detection sensor, a location determination sensor, a movement sensor, and a sensor connected to one or more medicine containers storing medication consumed by the subject.

3. The system of claim 1, wherein the second plurality of parameters include information relating to at least one of medication intake by the subject, sleeping patterns of the subject, physical activity rate of the subject, blood pressure of the subject, weight of the subject, blood glucose level of the subject, physical state of the subject, and emotional state of the subject.

4. The system of claim 1, wherein the predetermined range comprises an expected range of values for the first and the second plurality of parameters.

5. The system of claim 4, wherein the processor is configured to determine the expected range based on historical measurements of the at least one parameter.

6. The system of claim 5, wherein the processor is configured to obtain the historical measurements of the at least one parameter by storing measurement of the at least one parameter at two or more instants of time.

7. The system of claim 5, wherein the processor is configured to determine a subject-specific expected range for the subject based on the historical measures associated with that subject.

8. The system of claim 1, wherein the processor is configured to monitor the first and second parameters and, in an event the first and second pluralities of parameters fall within the predetermined range, issue an all clear signal indicating that the subject is in a stable physiological condition.

9. The system of claim 1, wherein the processor is configured to issue the alarm signal to an entity capable of providing immediate assistance to the subject.

10. The system of claim 1, wherein the alarm signal is a reminder signal issued to the subject.

11. The system of claim 1, wherein the processor is configured to monitor the at least one parameter over a predetermined time interval.

12. The system of claim 11, wherein the predetermined time interval comprises at least one time interval during which the one or more sensors did not operate to detect the first plurality of parameters, and wherein the processor is further configured to estimate values of the plurality of parameters during the at least one time interval.

13. The system of claim 12, wherein the processor is processor is configured to determine a confidence interval for estimated values of the plurality of parameters and in an event at least one estimated value of a parameter falls outside of the predetermined range, issue the alarm signal.

14. The system of claim 13, wherein the alarm comprises a coaching message instructing the subject to take at least one action regarding their health.

15. A system comprising:
one or more interfaces each configured to detect a plurality of parameters relating to physiological condition of a subject; and
at least one processor coupled to the one or more interfaces, the processor being configured to monitor the plurality of parameters and, in an event at least one parameter falls outside of a predetermined range, issue an alarm signal indicating onset of an undesired event in the physiological condition of the subject.

16. The system of claim 15, wherein at least one interface comprises a sensor configured to detect a parameter relating to the physiological condition of a subject.

17. The system of claim 16, wherein the sensor comprises at least one of a heart rate detection sensor, a location determination sensor, a movement sensor, and a sensor connected to one or more medicine containers storing medication consumed by the subject.

18. The system of claim 15, wherein the plurality of parameters comprise information relating to at least one of medication intake by the subject, sleeping patterns of the subject, physical activity rate of the subject, blood pressure of the subject, weight of the subject, blood glucose level of the subject, physical state of the subject, and emotional state of the subject.

19. The system of claim 15, wherein the predetermined range comprises an expected range of values for the at least one parameter.

20. The system of claim 19, wherein the processor is configured to determine the expected range based on historical measurements of the at least one parameter.

21. The system of claim 20, wherein the processor is configured to obtain the historical measures of the at least one parameter by storing measurement of the at least one parameter at two or more instants of time.

22. The system of claim 21, wherein the processor is configured to determine a subject-specific expected range for the subject based on the historical measures.

23. The system of claim 15, wherein the processor is configured to monitor the plurality of parameters and, in an event the plurality of parameters fall within the predetermined range, issue an all clear signal indicating that the subject is in a stable physiological condition.

24. The system of claim 15, wherein the processor is configured to issue the alarm signal to an entity capable of providing immediate assistance to the subject.
